(19) United States
(12) Patent Application Publication
(10) Pub. No.: US 2017/0197068 A1
(43) Pub. Date: Jul. 13, 2017

(54) Medication Dispensing Device and a Medication Dispensing Assembly

(71) Applicant: Raumedic AG, Münchberg (DE)

(72) Inventor: Dominik Erhard, Kulmbach (DE)

(21) Appl. No.: 15/403,402

(22) Filed: Jan. 11, 2017

(30) Foreign Application Priority Data
Jan. 11, 2016 (DE) 10 2016 200 211.2

(51) Int. Cl. A61M 31/00; A61F 6/14; A61M 39/22

(52) U.S. Cl. CPC A61M 31/002; A61M 39/22; A61F 6/14; A61M 2230/50; A61M 2205/3576; A61M 2230/208; A61M 2230/005

(57) ABSTRACT

The invention relates to a medication dispensing device for dispensing a medication inside a living being, having a housing, a medication storage unit for storing a medication, disposed inside the housing, wherein the medication storage unit has a wall that is flexible, at least in sections, at least one medication dispensing orifice (16) for dispensing the medication, disposed in an outer wall of the housing, and a swelling element that is in contact with the flexible wall of the medication storage unit, for pressing a quantity of the medication out of the medication storage unit, wherein a liquid storage element is provided, which has a liquid conveyance connection to the swelling element.

### FIELD OF THE INVENTION

The contents of the German patent application DE 10 2016 200 211.2 are referenced herein.

The invention relates to a medication dispensing device as well as a medication dispensing assembly.

### BACKGROUND OF RELATED ART

Medication dispensing devices are known from EP 2 464 414 B1, WO 2012/128 497 A2 and EP 2 624 907 B1. DE 697 23 589 T2 discloses an osmotic administration system, and a method for increasing the performance and improving the initial effects of osmotic administration systems. DE 103 00 896 A1 describes an automatic conveyor device for conveying a medium, in particular insulin, having an adjustable dispensing characteristic. GB 2 347 081 A1 describes a device for administering a beneficial substance to an animal U.S. Pat. No. 5,869,078 describes an implantable medication dispensing device.

### SUMMARY OF THE INVENTION

One object of the invention is to provide a medication dispensing device, which ensures a releasing of a medication over the entire application period.

This object is achieved with the medication dispensing device having the features of claim 1.

It has been acknowledged in accordance with the invention that a swelling element is to be provided, which is in contact with a flexible wall of a medication storage unit. When a quantity of a liquid is received therein, the swelling element expands, resulting in a compression of the medication storage unit. As a result, a quantity of a medication is pressed out of the medication storage unit. An internal or external component of the medication dispensing device is used as the liquid storage element. The liquid storage element may be used for storing water or demineralized water.

The housing is advantageously composed of a biocompatible material, in particular a silicone.

The term “medication” used in this application is not to be understood to be limiting. The term “medication” refers to both a single medical substance as well as a medicinal composition.

The medication is preferably present in the form of a powder, in the form of a gel, in particular, or in the form of a liquid, in particular.

The medication dispensing device can be designed as a temporary implant. The medication dispensing device can be provided as a whole for administration to the living being, in particular for oral administration.

In a medication dispensing device according to claim 2, an electronic unit is provided inside the housing. This results in a compact device.

With a medication dispensing device according to claim 3, a valve is provided, disposed between the swelling element and the liquid storage element. Advantageously, it is possible to control the amount of liquid provided by the liquid storage unit by this means.

With a medication dispensing device according to claim 4, the valve is connected to the electronic unit for signal transmission. Advantageously, this enables an opening and closing of the valve.

With a medication dispensing device according to claim 5, the swelling element 1 is a liquid absorber. As a result, a structurally simple and cost efficient medication dispensing device is created.

With a medication dispensing device according to claim 6, the swelling element 1 is a liquid super-absorber.

Advantageously, the liquid super-absorber is made of a copolymer, e.g. acrylic acid and sodium acrylate. The copolymer is advantageously available in the form of a granulate.

Advantageously, there is a higher osmotic pressure in the liquid super-absorber, such that there is no need for an active conveyance of the liquid, e.g. by means of a pump or a hydrogen generator.

In an advantageous embodiment, the liquid super-absorber in granulate form is disposed in a membrane. Advantageously, this is a flexible membrane.

With a medication dispensing device according to claim 7, the electronic unit has at least one of the following components:
at least one sensor for measuring a body parameter of a living being,
a control element for controlling a valve,
a data transmission element for transmitting data to an external device and/or
a data storage unit for storing data.

Advantageously, the data transmission element is designed for wireless transmission of data, in particular the body parameters of the being measured by the sensors.

Advantageously, the electronic unit is assigned an energy storage element for storing electric energy, wherein the energy storage element provides energy for the components of the electronic unit as well as for the opening and closing of the valve.

With a medication dispensing device according to claim 8, at least one sensor is provided for measuring a pH value of a body fluid, a body temperature, a blood value, in particular a glucose level, or suchlike.

With a medication dispensing device according to claim 9, the liquid storage element is disposed inside the housing.

With a medication dispensing device according to claim 10, the liquid storage unit is disposed outside the housing.

Another object of the invention is to provide a medication dispensing assembly, suitable for at least temporary placement of a medication dispensing device in a being.

This object is achieved with a medication dispensing assembly having the features of claim 11.

It is useful to provide an outer housing having at least one further medication dispensing orifice, wherein a medication dispensing device can be disposed in the outer housing.

Advantageously, the outer housing is composed of a biocompatible material, in particular silicone.

With a medication dispensing assembly according to claim 12, the at least one medication dispensing orifice of the medication dispensing device and the at least one further medication dispensing orifice of the medication dispensing assembly are disposed such that they at least partially overlap.

With a medication dispensing assembly according to claim 13, the medication dispensing device is secured in the outer housing of the medication dispensing assembly by means of an adhesive. Advantageously, the adhesive is a sealing adhesive.

Depending on the application of the medication dispensing assembly, it may be useful to dispose the liquid storage element outside the medication dispensing device and inside the outer housing of the medication dispensing assembly, in accordance with claim 14, or to dispose the liquid storage element outside the medication dispensing device and outside the outer housing of the medication dispensing assembly, in accordance with claim 15.

Advantageously, with the medication dispensing assembly according to claim 15, the liquid storage element is connected with an adhesive bond to the medication dispensing assembly in a sealed manner

### BRIEF DESCRIPTION OF THE DRAWINGS

And exemplary embodiment can be explained in greater detail based on FIGS. 1 to 3. Therein:

FIG. 1 shows a schematic depiction of the medication dispensing device in the initial state,

FIG. 2 shows a schematic depiction of the medication dispensing device in an expanded state of the swelling element,

FIG. 3 shows a schematic depiction of the medication dispensing assembly.

### DETAILED DESCRIPTION OF THE PREFERRED EMBODIMENTS

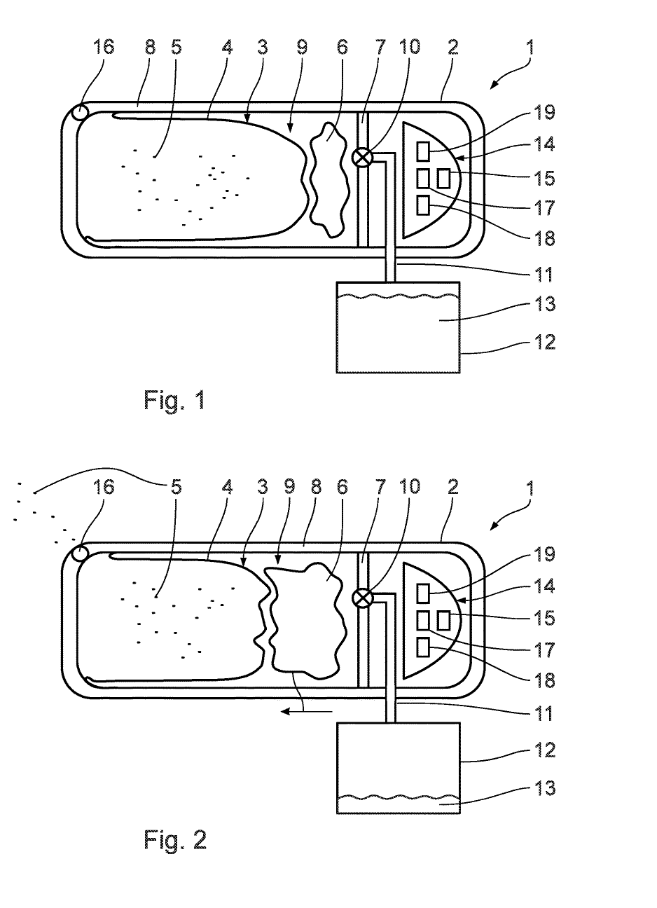

FIG. 1 shows a medication dispensing device 1 having a housing 2. A medication storage unit 3 is disposed inside the housing 2. The medication storage unit 3 has a wall 4 that is flexible, at least in sections, for receiving a medication 5. The flexible wall 4 is in contact with a swelling element 6. A housing inner wall 7 and a housing outer wall 8 form a housing chamber 9 in which the medication storage unit 3 and the swelling element 6 are disposed. The swelling element 6 is disposed thereby between the flexible wall 4 and the housing inner wall 7. A valve 10 is disposed in the housing inner wall 7, which is connected to the liquid storage element 12 via a supply line 11.

The liquid storage element 12 is filled with a liquid 13, in particular water, in particular demineralized water. The liquid storage element 12 is disposed outside the housing 2 in the illustrated exemplary embodiment. Depending on the application of the medication dispensing unit, the liquid storage element 12 can also be disposed inside the housing 2.

An electronic unit 14 is disposed in a region of the housing inner wall 7 separated from the swelling element 6. The electronic unit 14 has a control element 15, which is connected to the valve such that signals can be transmitted therebetween. The control element 15 is used to open and close the valve 10.

When the valve 10 is opened, the liquid 13 flows into the housing chamber 9 due to the osmotic pressure of the swelling element 7, such that the liquid 13 is absorbed by the swelling element 6. When the swelling element 6 absorbs the liquid 13, it expands, resulting in a compression of the medication storage unit 3, such that the medication 5 is discharged via a medication dispensing orifice 16.

The swelling element 6 is preferably a liquid absorber, in particular a liquid super-absorber. The liquid super-absorber is a copolymer, in particular a combination of an acrylic acid and a sodium acrylate.

When dry, the liquid super-absorber is preferably present as a granulate. When the valve 10 is opened, the liquid 13 flows into the housing chamber 9, such that it is absorbed by the granulate liquid super-absorber, and expands as a result of receiving the liquid, and forms a hydrogel. Because of the expansion of the liquid super-absorber, the flexible wall 4 of the medication storage unit 3 is compressed. As a result, the medication 5 is pressed out of the medication storage unit 3, and discharged into the living being via the medication dispensing orifice 16.

The control element 15 has a signal transmission connection with other components of the electronic unit 14. One of these components can be a sensor 17 for measuring a body temperature of the being, a data transmission element 18 for transmitting data to an external device and/or a data storage element 19 for storing data. This list is not exclusive, and further components that are designed to enable a controllable dispensing of medication may be provided. These shall be explained in greater detail below, based on a concrete example.

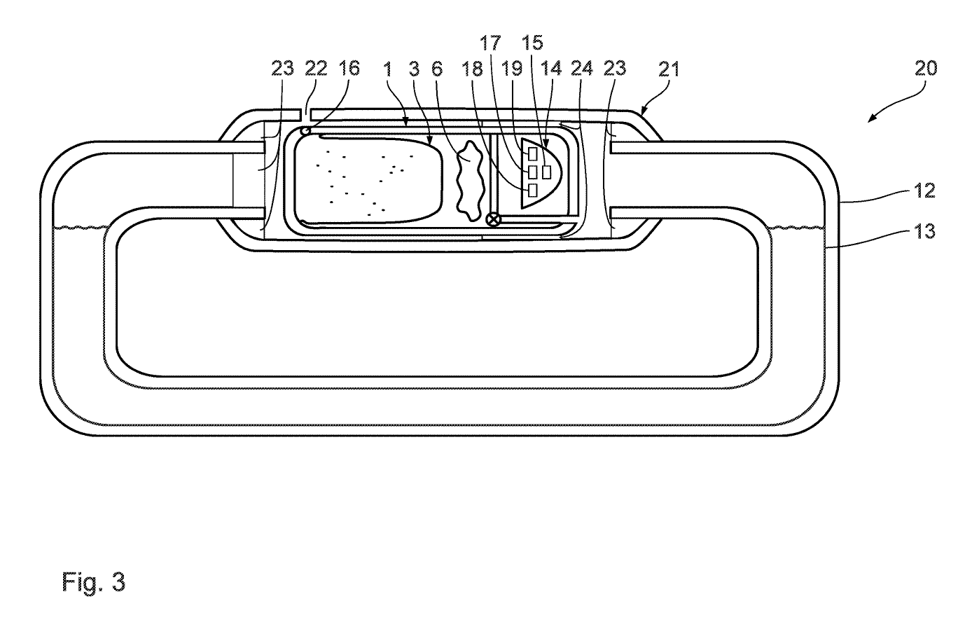

FIG. 3 shows a medication dispensing assembly 20 for at least temporary placement of a medication dispensing device according to FIGS. 1 and 2. The medication dispensing assembly 20 is composed of an outer housing 21 that has at least one further medication dispensing orifice 22, and a medication dispensing device 1 disposed in the outer housing 21. Furthermore, a liquid storage element 12 containing a liquid 13 is provided. The liquid storage element 12 is secured on the outer housing 20 at adhesive points 23 in a sealing manner. The medication dispensing device 1 is disposed in the outer housing 21 such that the medication dispensing orifice 16 of the medication dispensing device 1 at least partially overlaps the medication dispensing orifice 22 of the outer housing 21. The medication dispensing device 1 is secured in the outer housing 21 by means of an adhesive.

Depending on the application, the outer housing 21 can also enclose the liquid storage element.

In the exemplary embodiment illustrated according to FIG. 3, the liquid storage element 12 takes the form of a hose. This hose is designed such that it is sealed at one end against the medication dispensing device 1, while the other end of the liquid storage element 12 has a fluid conveying connection to the medication dispensing device 1. There is enough liquid 13 contained in the liquid storage element 12 that when the valve 10 is open, the swelling element 6 can accommodate enough liquid 13 to fully empty the medication dispensing unit 3.

One possible application of the medication dispensing assembly 20 is as an intra-vaginal ring, which can be placed in the vagina for numerous weeks. Contraceptive hormones, fertility hormones, insulin or suchlike can be released continuously there via the medication dispensing device 1. Depending on the application of the medication dispensing assembly and the embodiment of the medication dispensing device 1, a intermittent dispensing of the medication can also be provided. By way of example, a sensor 17 for determining a glucose level in the being can be provided in the electronic unit 14 of the medication dispensing device 1. This continuously measures the glucose level. These measured glucose levels can be stored in the data storage element 19, and advantageously transmitted in a wireless manner to an external device via the data transmission element 18. As soon as a threshold value is exceeded, a signal is sent to the control element 15, at which point the valve 10 is opened. By opening the valve 10, the liquid 13 is discharged from the liquid storage element 12 into the housing chamber 9, at which point the swelling element 6 expands, and a quantity of insulin is released from the medication storage unit 3. When the threshold value is no longer exceeded, another signal is sent to the control element 15, at which point the valve 10 closes. By this means, no more liquid 13 flows into the housing chamber 9, at which point the swelling element 6 no longer expands, and no more insulin is released. After the maximum application period has been reached, the medication dispensing assembly 20 can be removed, and replaced, if necessary.

In an alternative design of the medication dispensing device, it is designed in particular for oral administration. The medication dispensing device can be contained as a whole in a capsule that can be swallowed.

1. A medication dispensing device for administering a medication inside a living being, comprising:
a housing;
a medication storage unit for storing a medication, disposed inside the housing, wherein the medication storage unit has a wall that is flexible, at least in sections;
at least one medication dispensing orifice disposed in an outer wall of the housing, for dispensing the medication; and
a swelling element that is in contact with the flexible wall of the medication storage unit, for squeezing out a quantity of the medication from the medication storage unit; and
a liquid storage element, which has a liquid conveying connection to the swelling element.

2. The medication dispensing device according to claim 1, further comprising an electronic unit disposed inside the housing.

3. The medication dispensing device according to claim 2, further comprising a valve disposed between the swelling element and the liquid storage element, for controlling a quantity of a liquid that is to be discharged from the liquid storage element.

4. The medication dispensing device according to claim 3, wherein the valve is in signal transmitting contact with the electronic unit.

5. The medication dispensing device according to claim 1, wherein the swelling element is a liquid absorber.

6. The medication dispensing device according to claim 1, wherein the swelling element is a liquid super-absorber.

7. The medication dispensing device according to claim 2, wherein the electronic unit is comprised of at least one of:
(a) a sensor for measuring a body temperature of the being,
(b) a control element for controlling the valve,
(c) a data transmission element for transmitting data to an external device, and
(d) a data storage element for storing data.

8. The medication dispensing device according to claim 7, wherein a sensor is provided for measuring a pH value of a body fluid, a body temperature, a blood value, or suchlike.

9. The medication dispensing device according to claim 1, wherein the liquid storage element is disposed inside the housing.

10. The medication dispensing device according to claim 1, wherein the liquid storage element is disposed outside the housing.

11. A medication dispensing assembly for at least temporary placement of a medication dispensing device in a living being, comprising:
an outer housing having at least one further medication dispensing orifice; and
a medication dispensing device according to claim 1, disposed in the outer housing.

12. The medication dispensing assembly according to claim 11, wherein the at least one medication dispensing orifice of the medication dispensing device and the at least one further medication dispensing orifice of the medication dispensing assembly are disposed such that they at least partially overlap.

13. The medication dispensing assembly according to claim 11, wherein the medication dispensing device is secured in the outer housing of the medication dispensing assembly by means of an adhesive.

14. The medication dispensing assembly according to claim 11, wherein the liquid storage element is disposed outside the medication dispensing device and inside the outer housing of the medication dispensing assembly.

15. The medication dispensing assembly according to claim 11, wherein the liquid storage element is disposed outside the medication dispensing device and outside the outer housing of the medication dispensing assembly.

16. A medication dispensing device for administering a medication inside a living being comprising:
a housing comprised of a wall having an opening through which medication passes when dispensed;
a medication storage unit comprised of a flexible wall disposed within the housing;
a swelling element that operatively cooperates with the flexible wall of the medication storage unit that dispenses a quantity of medication from the medication storage unit when the swelling element swells;
a liquid dispensing arrangement in fluid-flow communication with the swelling element, the liquid dispensing arrangement comprised of a valve for controlling flow of liquid to the swelling element, and an electronic unit comprised of a valve control element for controlling operation of the valve and which is configured to control the quantity of medication dispensed from the medication storage unit by controlling the amount of liquid dispensed via the valve to the swelling element that swells up the swelling element.

17. The medication dispensing device according to claim 16 wherein the swelling element is disposed within the housing and in contact with the flexible wall of the medication storage unit, and wherein the liquid dispensing arrangement further comprises a liquid storage element that holds liquid conveyed through a liquid conveying connection to the swelling element when the valve is open.

18. The medication dispensing device according to claim 17 wherein the swelling element is a superabsorber and the liquid in the liquid storage element dispensed to swell up the swelling element is comprised of water.

19. The medication dispensing device according to claim 16 wherein the opening in the housing through which medication passes when dispensed comprises a dispensing orifice through which medication from the medication storage unit is dispensed when liquid swells up the swelling element.

20. The medication dispensing device according to claim 16 wherein the medication storage unit, the swelling element, and the valve of the liquid dispensing arrangement are disposed inside the housing.
